(19) United States
(12) Patent Application Publication
(10) Pub. No.: US 2026/0305915 A1
(43) Pub. Date: Oct. 8, 2026

(54) FIBER BRAGG GRATING FOR ENDOSCOPES

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Charles A. Baker, Rogers, MN (US); Fredrick Pelton, Akron, OH (US)

(21) Appl. No.: 19/482,161

(22) PCT Filed: May 8, 2024
(86) PCT No.: PCT/US2024/028359

§ 371 Date: Nov. 6, 2025

Related U.S. Application Data
(60) Provisional application No. 63/465,172, filed on May 9, 2023.
(60) Provisional application No. 63/558,352, filed on Feb. 27, 2024.

(51) Int. Cl. A61B 1/00; A61B 1/015; A61B 90/00; A61M 13/00

(52) U.S. Cl. CPC A61B 1/00097; A61B 1/00055; A61B 1/015; A61M 13/003; A61B 2090/064; A61B 2562/0266; A61M 2205/3306; A61M 2205/3344

(57) ABSTRACT

In a surgical system, an endoscope can extend distally toward a target site. A sensing optical fiber, extending to a distal portion of the endoscope, can include a fiber Bragg grating disposed at a distal portion of the sensing optical fiber. A sensing controller can direct sensing light distally along the sensing optical fiber such that at least some of the sensing light reflects from the fiber Bragg grating as reflected light. The sensing controller can perform optical frequency domain reflectometry on the reflected light to determine a parameter value, such as a pressure value or a temperature value, at the fiber Bragg grating. Processing circuitry can analyze the parameter value to determine that a parameter, such as a pressure or a temperature, at the target site satisfies a specified condition, such as being too high, and, in response, generate an alert data signal or perform a suitable task.

### CROSS-REFERENCE TO RELATED APPLICATIONS

This application claims the benefit of U.S. Provisional Application No. 63/465,172, filed May 9, 2023, and U.S. Provisional Application No. 63/558,352, filed Feb. 27, 2024, both of which are hereby incorporated by reference in their entireties.

### FIELD OF THE DISCLOSURE

This document relates generally to endoscopic systems, and more specifically relates to systems and methods for determining and controlling a distance between a tip of a medical device and a target.

### BACKGROUND OF THE DISCLOSURE

An operator, such as a physician, practitioner, or user, can use an endoscope to provide visual access to an internal location of a patient. The operator can insert an endoscope into a patient's body. The endoscope can deliver light to a target being examined, such as a target anatomy or object. The endoscope can collect light that is reflected from the object. The reflected light can carry information about the target being examined.

An endoscope can include a working channel. The operator can perform suction through the working channel. The operator can pass instruments, such as brushes, biopsy needles or forceps, through the working channel. The operator can perform minimally invasive surgery through the working channel, such as to remove unwanted tissue or foreign objects from the body of the patient.

An endoscope can use a laser or plasma system to perform laser therapy, such as ablation, coagulation, vaporization, fragmentation, lithotripsy, and others. In laser therapy, the operator can use the endoscope to deliver surgical laser energy to various target treatment areas, such as soft or hard tissue. In lithotripsy, the operator can use the endoscope to deliver surgical laser energy to break down calculi structures in the patient's kidney, gallbladder, ureter, or other stone-forming regions, or to ablate large calculi into smaller fragments.

### SUMMARY

In an example, a surgical system can comprise: an endoscope configured to extend distally toward a target site; a sensing optical fiber extending to a distal portion of the endoscope and including a fiber Bragg grating disposed at a distal portion of the sensing optical fiber; a sensing controller configured to: direct sensing light distally along the sensing optical fiber such that at least some of the sensing light reflects from the fiber Bragg grating as reflected light; perform optical frequency domain reflectometry on the reflected light; and determine, from the optical frequency domain reflectometry, a parameter value at the fiber Bragg grating; and processing circuitry configured to: analyze the parameter value; determine, from the analysis of the parameter value, that a parameter at the target site satisfies a specified condition; and generate an alert data signal in response to the determination that the parameter at the target site satisfies the specified condition.

In an example, a surgical system can comprise: an endoscope configured to extend distally toward a target site and provide insufflation media to the target site; a sensing optical fiber extending to a distal portion of the endoscope and including a fiber Bragg grating disposed at a distal portion of the sensing optical fiber; a sensing controller configured to: direct sensing light distally along the sensing optical fiber such that at least some of the sensing light reflects from the fiber Bragg grating as reflected light; perform optical frequency domain reflectometry on the reflected light; and determine, from the optical frequency domain reflectometry, a pressure value at the fiber Bragg grating; and processing circuitry configured to: compare the pressure value to a threshold pressure value; determine, from the comparison, that the pressure value exceeds a threshold pressure value; and in response to the determination that the that the pressure value exceeds the threshold pressure value, cause the endoscope to perform at least one of: provide an alert to a user to reduce a pressure of the insufflation media; or automatically reduce a pressure of the insufflation media.

In an example, a surgical system can comprise: an endoscope configured to extend distally toward a target site, provide therapeutic laser light to the target site, and provide a flushing agent to the target site, the endoscope including a cooling element configured to controllably cool the target site; a sensing optical fiber extending to a distal portion of the endoscope and including a fiber Bragg grating disposed at a distal portion of the sensing optical fiber; a sensing controller configured to: direct sensing light distally along the sensing optical fiber such that at least some of the sensing light reflects from the fiber Bragg grating as reflected light; perform optical frequency domain reflectometry on the reflected light; and determine, from the optical frequency domain reflectometry, a temperature value at the fiber Bragg grating; and processing circuitry configured to: compare the temperature value to a threshold temperature value; determine, from the comparison, that the temperature value exceeds a threshold temperature value; and in response to the determination that the that the temperature value exceeds the threshold temperature value cause the endoscope to perform at least one of: provide an alert to a user that the temperature at the target site may be too high; automatically reduce a power of the therapeutic laser light; automatically increase a flow rate of the flushing agent; or automatically activate the cooling element.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 shows a side-view schematic drawing of an example of a surgical system

FIG. 2 shows a flow chart of an example of a method for operating an endoscope.

FIG. 3 shows a flow chart of an example of a method for operating an endoscope.

FIG. 4 shows a flow chart of an example of a method for operating an endoscope.

FIG. 5 shows a schematic diagram of an example of a computer-based clinical decision support system that is configured to provide a value of distance based on an optical property associated with the light from the target site.

### DETAILED DESCRIPTION

In a surgical system, an endoscope can extend distally toward a target site. A sensing optical fiber, extending to a distal portion of the endoscope, can include a fiber Bragg grating disposed at a distal portion of the sensing optical fiber. A sensing controller can direct sensing light distally along the sensing optical fiber such that at least some of the sensing light reflects from the fiber Bragg grating as reflected light. The sensing controller can perform optical frequency domain reflectometry on the reflected light to determine a parameter value, such as a pressure value or a temperature value, at the fiber Bragg grating. Processing circuitry can analyze the parameter value to determine that a parameter, such as a pressure or a temperature, at the target site satisfies a specified condition, such as being too high. In response, the processing circuitry can generate an alert data signal and/or perform another task, such as automatically adjusting or reducing a pressure of insufflation media, automatically adjusting or reducing a flow rate of a flushing agent, or automatically activating a cooling element or a heating element.

For the purposes of this document, the term “automatically” can signify that a particular action may be initiated by processing circuitry. For example, the term “automatically” can signify that a particular action can be taken without receiving instructions from a user to initiate the particular action. The term “automatically” may include prompting a user for confirmation and obtaining confirmation from the user to proceed with the particular action. For example, the processing circuitry may automatically calculate or automatically determine a new proposed parameter value, and may prompt a user to accept the proposed parameter value or confirm that proposed parameter value is acceptable.

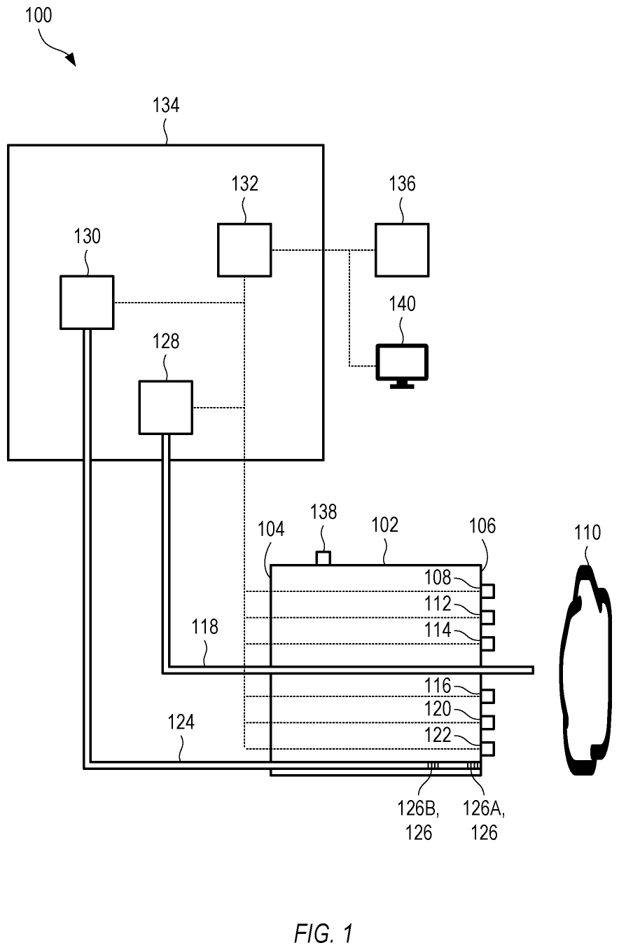

FIG. 1 shows a side-view schematic drawing of an example of a surgical system 100. The configuration of FIG. 1 is but one example of a surgical system; other configurations can also be used.

The surgical system 100 can include an endoscope 102. The endoscope 102 can include an elongated body portion extending between a proximal end 104 and a distal end 106. The precise shape of the elongated body portion can depend on the medical procedure for which the endoscope 102 was originally designed. For simplicity, the elongated body portion is shown as being a cylinder, and having a circular cross-section, taken orthogonal to the direction of elongation. Other suitable shapes can also be used. During use in a procedure, the endoscope 102 can extend distally toward a target site 110, such as a kidney stone. The endoscope 102 can include one or more light sources or light emitters 108 at the distal end 106 of the endoscope 102 to illuminate the target site 110. Examples of suitable light emitters 108 can include a light-emitting diode, such as a white light-emitting diode, an arc lamp, such as a xenon arc lamp, and others. The endoscope 102 can include one or more cameras or imaging sensors 112 at the distal end 106 of the endoscope 102 to capture an image, such as a real-time video image, of the illuminated target site 110. The endoscope 102 can include one or more heating elements 114, such as electrically resistive heaters, that can controllably heat the target site 110 or one or more other suitable regions on the endoscope 102. The endoscope 102 can include one or more cooling elements 116, such as thermoelectric coolers, that can controllably cool the target site 110 or one or more other suitable regions on the endoscope 102. The endoscope 102 can provide insufflation media, such as helium or carbon dioxide, via an insufflation media port 120, to the target site 110 to temporarily inflate the target site 110 during the procedure. The endoscope 102 can provide a flushing agent, such as saline, via a flushing agent port 122, to the target site 110 such as to help cool or remove particles, such as kidney stone fragments, that may be generated during the procedure.

The endoscope 102 can include various electrical connections that extend along a length of the endoscope 102, such as to electrically power the one or more light emitters 108, deliver data signals from the one or more imaging sensors 112, electrically power the one or more heating elements 114, electrically power the one or more cooling elements 116, and others. Although the electrical connections in FIG. 1 are shown as each extending proximally to a respective location at the proximal end 104 of the endoscope 102, in practice, the electrical connections can be grouped together so that the electrical connections can be made with a single connector at or near the proximal end 104 of the endoscope 102. For example, in practice, the electrical connections can extend along a single channel within the endoscope 102 and can fan out as needed at or near the distal end 106 of the endoscope 102.

The endoscope 102 can provide therapeutic laser light to the target site 110. The surgical system 100 can include at least one therapeutic optical fiber 118 to deliver the therapeutic laser light. The at least one therapeutic optical fiber 118 can be positionable to extend from the distal end 106 of the endoscope 102. The therapeutic optical fiber 118 can be configured to emit the therapeutic fiber light toward the target site 110, such as to be directed onto a kidney stone to perform dusting and/or fragmenting of the kidney stone. The surgical system 100 can include a therapeutic laser light source 128 that can generate the therapeutic laser light, direct the therapeutic laser light into a proximal portion of the therapeutic optical fiber 118, and direct the therapeutic laser light distally along a length of the therapeutic optical fiber 118 to emerge from a distal end of the therapeutic optical fiber 118 to form the therapeutic fiber light. The therapeutic laser light source 128 can include a thulium fiber laser, which can produce light at a wavelength of 1908 nm and/or 1940 nm. The therapeutic laser light source 128 can include a thulium:YAG (yttrium aluminum garnet) laser, which can produce light at a wavelength of 2010 nm. The therapeutic laser light source 128 can include a holmium:YAG laser, which can produce light at a wavelength of 2120 nm. The therapeutic laser light source 128 can include an erbium:YAG laser, which can produce light at a wavelength of 2940 nm. For these (and other) therapeutic laser light sources, the therapeutic laser light can have a wavelength or wavelengths in a portion of the electromagnetic spectrum at which water (a major component of tissue) has a relatively high absorption. During a procedure, the tissue can absorb the therapeutic laser light, can heat locally to a relatively high temperature, and can break apart due to local thermal strains within the tissue.

The surgical system 100 can include a sensing optical fiber 124 that can extend to a distal portion of the endoscope 102. The sensing optical fiber 124 can include a fiber Bragg grating 126 disposed at a distal portion of the sensing optical fiber.

The sensing optical fiber 124 may include more than one fiber Bragg grating 126 along a length of the sensing optical fiber 124. For example, the sensing optical fiber 124 can include a first fiber Bragg grating 126A and a second fiber Bragg grating 126B located proximal to the first fiber Bragg grating 126A. Using multiple fiber Bragg gratings 126 along the sensing optical fiber 124 can allow the surgical system to determine one or more physical conditions at the locations of the fiber Bragg gratings 126. For example, the sensing optical fiber 124 can be routed in the endoscope 102 to have the first fiber Bragg grating 126A at or near the distal end 106 of the endoscope 102, to determine a physical condition at or near the target site 110, and the second fiber Bragg grating 126B, to determine a physical condition at or near a component of the endoscope 102, such as the handle. Other locations can also be used.

Alternatively, or in addition, using multiple fiber Bragg gratings 126 along the sensing optical fiber 124 can allow the surgical system 100 to compare the physical conditions at two different locations. For example, the surgical system 100 can use measurements of pressure or fluid pressure, taken at two different locations along a fluid channel, to determine whether the fluid channel is blocked.

The surgical system 100 can include a sensing controller 130 coupled to the sensing optical fiber 124. The sensing controller 130 can direct sensing light distally along the sensing optical fiber 124 such that at least some of the sensing light reflects from the fiber Bragg grating 126 as reflected light. The sensing controller 130 can perform optical frequency domain reflectometry on the reflected light. The sensing controller 130 can determine, from the optical frequency domain reflectometry, a parameter value at the fiber Bragg grating 126. Suitable parameter values can include pressure values, temperature values, and others. For sensing optical fibers 124 that include multiple fiber Bragg gratings 126, the sensing controller 130 can determine the parameter values at some or all of the locations of the fiber Bragg gratings 126, and can do so essentially simultaneously via the optical frequency domain reflectometry. The sensing controller 130 can use optical frequency domain reflectometry (OFDR) with the sensing optical fiber 124 to determine a temperature value and/or a (fluid) pressure value at the location of the fiber Bragg grating 126, or at one or more fiber Bragg gratings 126 disposed along the sensing optical fiber 124. The sensing controller 130 can include a variable frequency laser beam coupled to an optical interferometer. The sensing controller 130 can split light from the variable frequency laser beam between a reference arm and a measurement arm of an interferometer. In the optical path of the measurement arm, the sensing controller 130 can further split the light to propagate distally along a length of the sensing optical fiber 124 that includes a fiber Bragg grating 126, and return proximally along the sensing optical fiber 124. Light in the measurement arm can interfere with light in the reference arm to form an interference pattern. The sensing controller 130 can include an optical detector that can detect the interference pattern. Other suitable configurations can also be used to determine temperature and/or pressure at one or more fiber Bragg gratings 126 disposed along the sensing optical fiber 124.

For example, the polarization-maintaining fiber can have a cross-section that includes a core at its center, and two holes on opposite sides of the core. The holes can define two hollow (or gas-filled) passages that extend along a length of the fiber on opposite sides of the core. The holes can optionally be circular in cross-section. The holes induce a birefringence in the fiber, such that a cross-sectional axis extending through centers of the holes can define a slow axis, and a cross-sectional axis extending between the holes (e.g., with the holes on opposite sides of the axis) can define a fast axis. The core can optionally have a cross-section that is elongated along the fast axis. In the polarization-maintaining fiber, light launched into the fiber with a linear polarization aligned with the fast axis can emerge from the fiber with a linear polarization that is aligned with the fast axis. Similarly, light launched into the fiber with a linear polarization aligned with the slow axis can emerge from the fiber with a linear polarization that is aligned with the slow axis.

In addition to providing the birefringence in the fiber, the holes can provide a relatively high sensitivity to pressure along the fast axis and a relatively low sensitivity to pressure along the slow axis. For example, a change in pressure may shift a wavelength at which a fiber Bragg grating 126 is reflective by a relatively small wavelength shift for the slow axis and a relatively large wavelength shift for the fast axis.

In contrast to pressure, the sensitivity to temperature may be the same along the slow and fast axes. For example, a change in temperature value may shift a wavelength at which the fiber Bragg grating 126 is reflective by the same wavelength shift for both the slow axis and the fast axis.

Because the polarization-maintaining fiber (having holes therethrough) shows a direction-dependent difference in pressure sensitivity but not in temperature sensitivity, the polarization-maintaining fiber (having holes therethrough) can allow the surgical system 100 to separate the effects of temperature from pressure, and therefore obtain more accurate values of both temperature and pressure. Specifically, the sensing controller 130 can direct first sensing light, which is linearly polarized along the slow axis of the sensing optical fiber 124, distally along the sensing optical fiber 124, and direct second sensing light, which is linearly polarized along the fast axis of the sensing optical fiber 124, distally along the sensing optical fiber 124. The sensing controller 130 can take two measurements of pressure, with one for the first sensing light and one for the second sensing light. The sensing controller 130 can use a difference in value between the two pressure measurements, optionally with one or both of the pressure measurements, and optionally with one or both of the temperature measurements, to accurately determine the pressure values at the fiber Bragg gratings 126. In other words, detecting changes in the wavelength difference between the reflected fast axis and slow axis signals can allow the surgical system 100 to detect and measure pressure changes at the fiber Bragg gratings, and do so independent of temperature variations and strain at the fiber Bragg gratings. The surgical system 100 can use the OFDR techniques described herein to perform the pressure and temperature measurements and can optionally take a set of measurements for the fast axis and a another set of measurements for the slow axis to more accurately determine the pressure and/or temperature.

The surgical system 100 can include processing circuitry 132 coupled to the sensing controller 130. The processing circuitry 132 may be referred to as a controller. The processing circuitry 132 may be implemented purely in software. The processing circuitry 132 may be implemented purely in hardware. In some examples, processing circuitry 132 may be implemented as a combination of software and hardware. The processing circuitry 132 may be implemented on a single processor. The processing circuitry 132 may be implemented on multiple processors. The multiple processors may be housed in a common housing, such as housing 134. In some examples, at least two of the multiple processors may be spaced apart in different housings. The housing 134 can house one or more of the processing circuitry 132, the sensing controller 130, or the therapeutic laser light source 128. The processing circuitry 132 can include one or more processors, memory containing instructions that are executable by the one or more processors to cause the one or more processors to perform operations. Examples of such operations are detailed below.

The processing circuitry 132 can analyze the parameter value. The processing circuitry 132 can determine, from the analysis of the parameter value, whether a parameter at the target site 110 satisfies a specified condition. Suitable parameters can include pressure, temperature, and other physical conditions. Suitable specified conditions can include a pressure being too high or too low, a pressure gradient (such as obtained from two or more locations along the sensing optical fiber 124) being too high or too low, a rate of change of pressure (such as from repeated measurements over time of a time-varying pressure) being too high or too low, a temperature being too high or too low, a temperature gradient (such as obtained from two or more locations along the sensing optical fiber 124) being too high or too low, a rate of change of temperature (such as from repeated measurements over time of a time-varying temperature) being too high or too low, and others. Determining whether a parameter value is too high can include determining that the parameter value is greater than a specified threshold value. Determining that a parameter value is too low (or too high) can include determining that the parameter value is less than (or greater than) a specified threshold value. Other criteria can also be used.

The processing circuitry 132 can take one or more actions in response to the determination that the parameter at the target site 110 (or elsewhere along the sensing optical fiber 124) satisfies the specified condition.

An example of a suitable action can include generating an alert data signal 136, such as to alert a user, device, or process. The alert data signal 136 can alert a user that the pressure at the target site 110 may be too high. The alert data signal 136 can alert a user that the pressure at the target site 110 may be changing too quickly. The alert data signal 136 can alert a user that an insufflation media channel may be blocked. The alert data signal 136 can alert a user to adjust a pressure of the insufflation media. The alert data signal 136 can alert a user that the temperature at the target site 110 may be too high. The alert data signal 136 can alert a user that the temperature at the target site 110 may be changing too quickly. The alert data signal 136 can alert that the temperature at the target site 110 may be changing too quickly. The alert data signal 136 can alert that a component of the endoscope 102 may be too hot, and may instruct to take one or more actions to avoid damage to the component. Other suitable alert data signals can also be used. The alerts may be provided visually, such as via one or more indicator lights 138 or indicator signs on the endoscope 102, or on a graphical user display 140 coupled to the endoscope 102 or the processing circuitry 132. Alternatively or in addition, the alerts may be provided aurally, such as by a speaker on or in the endoscope 102, or on the graphical user display 140 coupled to the endoscope 102 or the processing circuitry 132.

Other suitable actions can include automatically adjusting a pressure of the insufflation media, automatically reducing a flow rate of the flushing agent, automatically increasing a flow rate of the flushing agent, automatically reducing a power of the therapeutic laser light, automatically activating one or more cooling elements 116, automatically activating one or more heating elements 114, and others.

More specific examples of parameters and parameter values, specified conditions, and suitable actions are described below.

In a first example, in which the parameter is a pressure and the parameter value is a pressure value, the processing circuitry 132 can, in response to the determination that the pressure at the target site 110 satisfies the specified condition, cause the endoscope 102 to provide an alert to a user, device, or process to adjust a pressure of the insufflation media. The processing circuitry 132 can, in response to the determination that the pressure at the target site 110 satisfies the specified condition, cause the endoscope 102 to automatically adjust a pressure of the insufflation media.

In a second example, in which the parameter is a pressure and the parameter value is a pressure value, the processing circuitry 132 can analyze the pressure value by comparing the pressure value to a threshold pressure value. The specified condition can be that the pressure value exceeds the threshold pressure value. The alert data signal 136 can alert a user, device, or process that the pressure at the target site 110 may be too high.

In a third example, in which the parameter is a pressure and the parameter value is a pressure value, the processing circuitry 132 can, in response to the determination that the pressure value exceeds the threshold pressure value, automatically reduce a flow rate of the flushing agent.

In a fourth example, in which the parameter is a pressure and the parameter value is a pressure value, the processing circuitry 132 can analyze the pressure value by comparing a rate of change of the pressure value to a specified criterion or condition, such as to a threshold pressure slope value. The specified condition can be that the rate of change of the pressure value exceeds the threshold pressure slope value. The alert data signal 136 can alert a user, device, or process that the pressure at the target site 110 may be changing too quickly.

In a fifth example, the sensing optical fiber 124 may include more than one fiber Bragg grating 126. For example, the parameter can be a pressure. The fiber Bragg grating 126 can be a first fiber Bragg grating 126A. The sensing optical fiber 124 can include a second fiber Bragg grating 126B located proximal to the first fiber Bragg grating 126A. The sensing controller 130 can determine, from the optical frequency domain reflectometry, a first pressure value at the first fiber Bragg grating 126A and a second pressure value at the second fiber Bragg grating 126B. The processing circuitry 132 can analyze the first pressure value and the second pressure value by comparing a difference between the first pressure value and the second pressure value to a specified criterion, such as to a threshold pressure difference. The processing circuitry 132 can determine, from the analysis of the first pressure value and the second pressure value, that the pressure at the target site 110 exceeds a threshold pressure difference. The alert data signal 136 can alert a user, device, or process that an insufflation media channel may be blocked.

In a sixth example, in which the parameter is a temperature and the parameter value is a temperature value, the processing circuitry 132 can analyze the temperature value by comparing the temperature value to a specified condition such as a threshold temperature value. The specified condition can be that the temperature value exceeds the threshold temperature value. The alert data signal 136 can alert a user, device, or process that the temperature at the target site 110 may be too high.

In a seventh example, in which the parameter is a temperature and the parameter value is a temperature value, the processing circuitry 132 can, in response to the determination that the temperature value meets a specified condition, e.g., exceeds the threshold temperature value, automatically reduce a power of the therapeutic laser light.

In an eighth example, in which the parameter is a temperature and the parameter value is a temperature value, the processing circuitry 132 can, in response to the determination that the temperature value meets a specified condition, e.g., exceeds the threshold temperature value, automatically increase a flow rate of the flushing agent.

In a ninth example, in which the parameter is a temperature and the parameter value is a temperature value, the processing circuitry 132 can, in response to the determination that the temperature value meets a specified condition, e.g., exceeds the threshold temperature value, automatically activate the cooling element 116.

In a tenth example, in which the parameter is a temperature and the parameter value is a temperature value, the processing circuitry 132 can analyze the temperature value by comparing the temperature value to a specified criterion, e.g., a threshold temperature value. The specified condition can be that the temperature value is below the threshold temperature value. The processing circuitry 132 can, in response to the determination that the temperature value is below the threshold temperature value, automatically activate the heating element 114.

In an eleventh example, in which the parameter is a temperature and the parameter value is a temperature value, the processing circuitry 132 can analyze the temperature value by comparing a rate of change of the temperature value to a specified criterion, e.g., a threshold temperature slope value. The specified condition can be that the rate of change of the temperature value exceeds the threshold temperature slope value. The alert data signal 136 can alert a user, device, or process that the temperature at the target site 110 may be changing too quickly.

In a twelfth example, the sensing optical fiber 124 may include more than one fiber Bragg grating 126. For example, the fiber Bragg grating can be a first fiber Bragg grating 126A. The sensing optical fiber 124 can include a second fiber Bragg grating 126B located proximal to the first fiber Bragg grating 126A. The sensing controller 130 can determine, from the optical frequency domain reflectometry, a first temperature value at the first fiber Bragg grating and a second temperature value at the second fiber Bragg grating. The processing circuitry 132 can analyze the first temperature value and the second temperature value. The processing circuitry 132 can determine, from the analysis of the second temperature value, that a temperature at a component of the endoscope 102 satisfies a specified condition. For example, the temperature at the component may exceed a specified threshold temperature for the component. As a specific example, the alert data signal 136 can alert a user, device, or process that the temperature at the component may be too hot, and may instruct to take one or more actions to avoid damage to the component.

The twelve specific examples of parameters and parameter values, specified conditions, and suitable actions are but mere examples. Other suitable combinations of parameters and parameter values, specified conditions, and suitable actions can also be used.

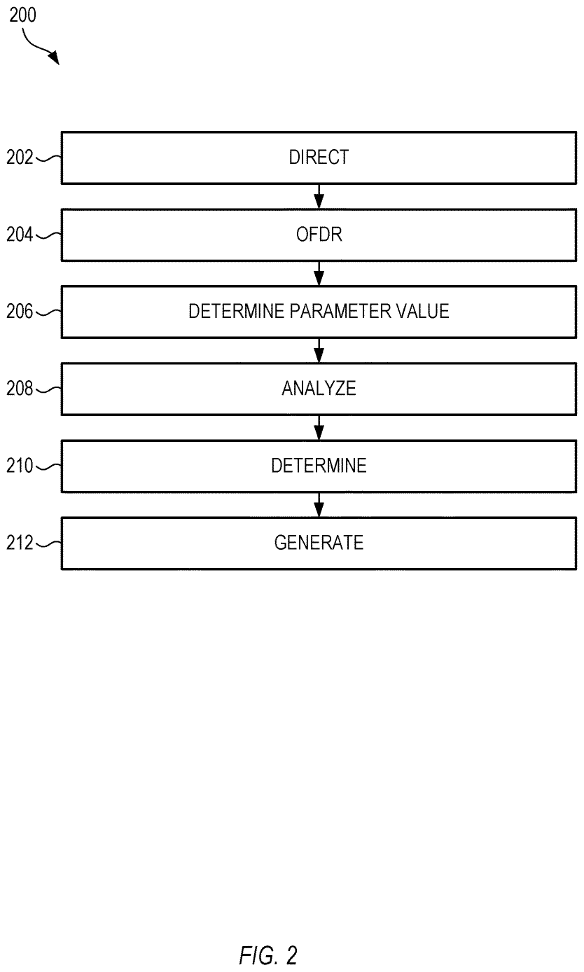

FIG. 2 shows a flow chart of an example of a method 200 for operating a surgical system, such as surgical system 100 (FIG. 1). In the surgical system, an endoscope, such as endoscope 102, can extend distally toward a target site, such as target site 110. A sensing optical fiber, such as sensing optical fiber 124, can extend to a distal portion of the endoscope and can include a fiber Bragg grating, such as fiber Bragg grating 126, disposed at a distal portion of the sensing optical fiber. The method 200 can be executed by the surgical system 100, or by another suitable surgical system. For example, the method 200 can be executed by a monopolar or bipolar radiofrequency device, an ultrasonic stone breaking device, an ultrasonic device for tissue modification, a combined energy device, a cold plasma type device, a diagnostic device such as a needle sampler where the device uses insufflation for creating an improved view of the target site, a diagnostic device that uses cryogenic or radiofrequency energy to capture the sample, and others. The method 200 is but one method for operating a surgical system; other suitable methods can also be used.

At operation 202, a sensing controller, such as sensing controller 130, can direct sensing light distally along the sensing optical fiber such that at least some of the sensing light reflects from the fiber Bragg grating as reflected light.

At operation 204, the sensing controller can perform optical frequency domain reflectometry (OFDR) on the reflected light.

At operation 206, the sensing controller can determine, from the optical frequency domain reflectometry, a parameter value at the fiber Bragg grating.

At operation 208, processing circuitry, such as processing circuitry 132, can analyze the parameter value.

At operation 210, the processing circuitry can determine, from the analysis of the parameter value, that a parameter at the target site satisfies a specified condition.

At operation 212, the processing circuitry can generate an alert data signal, such as alert data signal 136, in response to the determination that the parameter at the target site satisfies the specified condition.

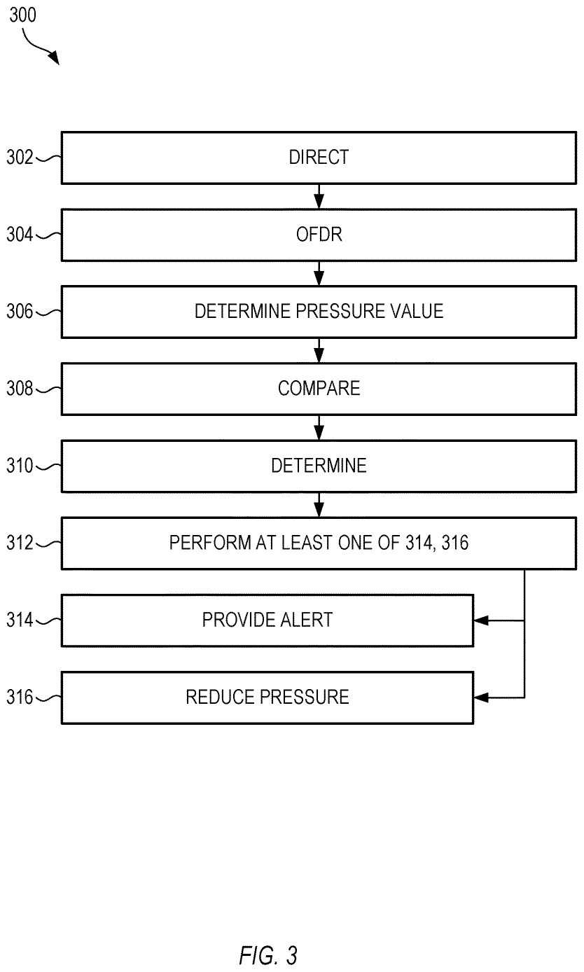

FIG. 3 shows a flow chart of an example of a method 300 for operating a surgical system, such as surgical system 100 (FIG. 1). In the surgical system, an endoscope, such as endoscope 102, can extend distally toward a target site, such as target site 110, and provide insufflation media to the target site. A sensing optical fiber, such as sensing optical fiber 124, can extend to a distal portion of the endoscope and can include a fiber Bragg grating, such as fiber Bragg grating 126, disposed at a distal portion of the sensing optical fiber. The method 300 can be executed by the surgical system 100, or by another suitable surgical system. The method 300 is but one method for operating a surgical system; other suitable methods can also be used.

At operation 302, a sensing controller, such as sensing controller 130, can direct sensing light distally along the sensing optical fiber such that at least some of the sensing light reflects from the fiber Bragg grating as reflected light.

At operation 304, the sensing controller can perform optical frequency domain reflectometry (OFDR) on the reflected light.

At operation 306, the sensing controller can determine, from the optical frequency domain reflectometry, a pressure value at the fiber Bragg grating.

At operation 308, processing circuitry, such as processing circuitry 132, can compare the pressure value to a specified criterion, such as to a threshold pressure value.

At operation 310, the processing circuitry can determine, from the comparison, that the pressure value exceeds a threshold pressure value.

At operation 312, the processing circuitry can, in response to the determination that the that the pressure value exceeds the threshold pressure value, cause the endoscope to perform at least one of operations 314 or 316.

At operation 314, the endoscope can provide an alert to a user, device, or process to reduce a pressure of the insufflation media.

At operation 316, the endoscope can automatically reduce a pressure of the insufflation media.

The endoscope can further provide a flushing agent to the target site. The processing circuitry can, in response to the determination that the pressure value exceeds the threshold pressure value, automatically reduce a flow rate of the flushing agent.

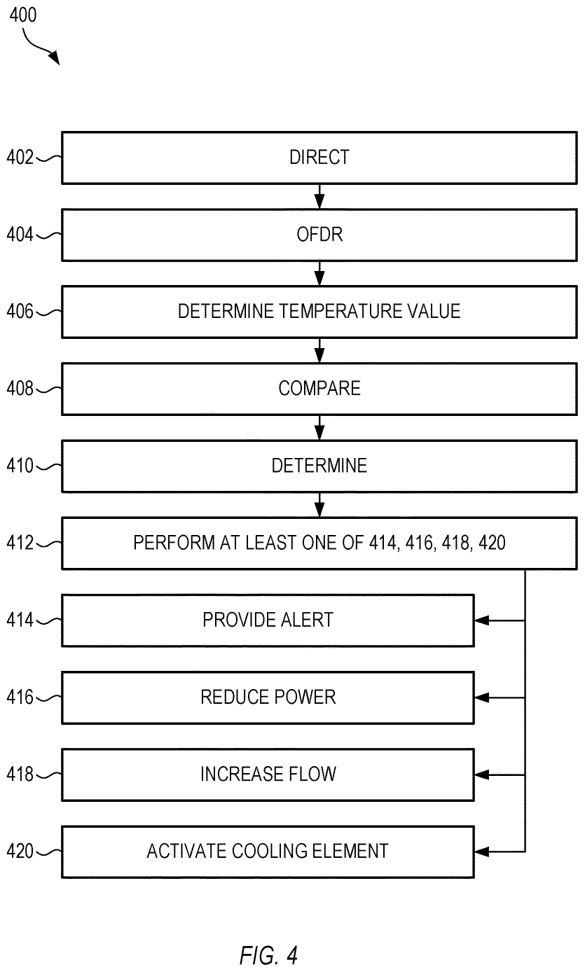

FIG. 4 shows a flow chart of an example of a method 400 for operating a surgical system, such as surgical system 100 (FIG. 1). In the surgical system, an endoscope, such as endoscope 102, can extend distally toward a target site, such as target site 110. The endoscope can provide therapeutic laser light to the target site. The endoscope can provide a flushing agent to the target site. The endoscope can include a cooling element that can controllably cool the target site. A sensing optical fiber, such as sensing optical fiber 124, can extend to a distal portion of the endoscope and can include a fiber Bragg grating, such as fiber Bragg grating 126, disposed at a distal portion of the sensing optical fiber. The method 400 can be executed by the surgical system 100, or by another suitable surgical system. The method 400 is but one method for operating a surgical system; other suitable methods can also be used.

At operation 402, a sensing controller, such as sensing controller 130, can direct sensing light distally along the sensing optical fiber such that at least some of the sensing light reflects from the fiber Bragg grating as reflected light.

At operation 404, the sensing controller can perform optical frequency domain reflectometry (OFDR) on the reflected light.

At operation 406, the sensing controller can determine, from the optical frequency domain reflectometry, a temperature value at the fiber Bragg grating.

At operation 408, processing circuitry, such as processing circuitry 132, can compare the temperature value to a specified criterion, such as a threshold temperature value.

At operation 410, the processing circuitry can determine, from the comparison, that the temperature value exceeds a threshold temperature value.

At operation 412, the processing circuitry can, in response to the determination that the that the temperature value exceeds the threshold temperature value cause the endoscope to perform at least one of operations 414, 416, 418, or 420.

At operation 414, the endoscope can provide an alert to a user, device, or process that the temperature at the target site may be too high.

At operation 416, the endoscope can automatically reduce a power of the therapeutic laser light.

At operation 418, the endoscope can automatically increase a flow rate of the flushing agent.

At operation 420, endoscope can automatically activate the cooling element.

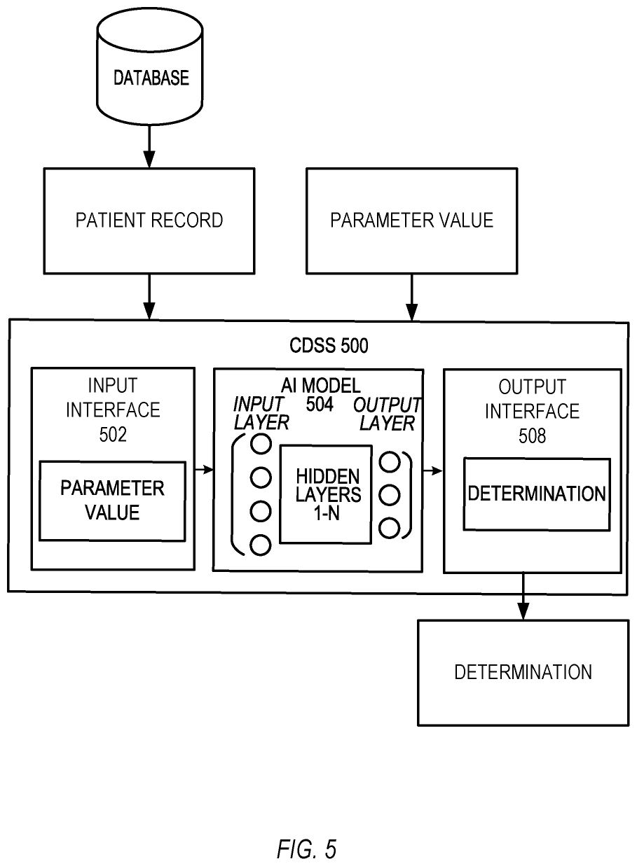

FIG. 5 shows a schematic diagram of an example of a computer-based clinical decision support system (CDSS) 500 that is configured to determine whether a parameter value, such as a pressure value or a temperature value, satisfies a specified condition. In various embodiments, the CDSS 500 includes an input interface 502 through which the parameter value which is specific to a patient is provided as input features to an artificial intelligence (AI) model 504, a processor which performs an inference operation in which the parameter value is applied to the AI model to determine whether the parameter value satisfies the specified condition, and a user interface (UI) or output interface 508 through which the determination is communicated to a user, e.g., a clinician.

In some embodiments, the input interface 502 may be a direct data link between the CDSS 500 and one or more medical devices, such as surgical system 100 or endoscope 102, which generate at least some of the input features. For example, the input interface 502 may transmit the parameter value directly to the CDSS during a therapeutic and/or diagnostic medical procedure. Additionally, or alternatively, the input interface 502 may be a classical user interface that facilitates interaction between a user and the CDSS 500. For example, the input interface 502 may facilitate a user interface through which the user may manually enter the parameter value. Additionally, or alternatively, the input interface 502 may provide the CDSS 500 with access to an electronic patient record from which one or more input features may be extracted. In any of these cases, the input interface 502 is configured to collect the parameter value in association with a specific patient on or before a time at which the CDSS 500 is used to assess the medical condition addressed by the surgical system 100 or endoscope 102, such as a kidney stone.

Based on one or more of the above input features, the processor, such as processing circuitry 132, performs an inference operation using the AI model to generate the determination. For example, input interface 502 may deliver the parameter value into an input layer of the AI model which propagates this input feature through the AI model to an output layer. The AI model can provide a computer system the ability to perform tasks, without explicitly being programmed, by making inferences based on patterns found in the analysis of data. AI model explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

The AI model may be trained continuously or periodically prior to performance of the inference operation by the processor, such as processing circuitry 132. Then, during the inference operation, the patient specific input features provided to the AI model may be propagated from an input layer, through one or more hidden layers, and ultimately to an output layer that corresponds to the determination.

The AI model can include a database, which can include data corresponding to a patient. The database can provide a patient record to the CDSS 500. The AI model can receive a parameter value from a sensor.

During and/or subsequent to the inference operation, the value of distance (Z) may be communicated to the user via the user interface (UI) and/or automatically cause an actuator or an alarm connected to the processor to perform a desired action. For example, the processor can cause the actuator to move the optical fiber with respect to the endoscope. Alternatively, the processor can cause the alarm to alert the practitioner.

The CDSS 500 can optionally be used to determine the action taken in response to a parameter value.

In the foregoing detailed description, the method and apparatus of the present disclosure have been described with reference to specific embodiments thereof. It will, however, be evident that various modifications and changes may be made thereto without departing from the broader spirit and scope of the present disclosure. The present specification and figures are accordingly to be regarded as illustrative rather than restrictive.

To further illustrate the device and related method disclosed herein, a non-limiting list of examples is provided below. Each of the following non limiting examples can stand on its own or can be combined in any permutation or combination with any one or more of the other examples.

In Example 1, a surgical system can comprise: an endoscope configured to extend distally toward a target site; a sensing optical fiber extending to a distal portion of the endoscope and including a fiber Bragg grating disposed at a distal portion of the sensing optical fiber; a sensing controller configured to: direct sensing light distally along the sensing optical fiber such that at least some of the sensing light reflects from the fiber Bragg grating as reflected light; perform optical frequency domain reflectometry on the reflected light; and determine, from the optical frequency domain reflectometry, a parameter value at the fiber Bragg grating; and processing circuitry configured to: analyze the parameter value; determine, from the analysis of the parameter value, that a parameter at the target site satisfies a specified condition; and generate an alert data signal in response to the determination that the parameter at the target site satisfies the specified condition.

In Example 2, the surgical system of Example 1 can optionally be configured such that: the parameter is a pressure; and the parameter value is a pressure value.

In Example 3, the surgical system of any one of Examples 1-2 can optionally be configured such that: the endoscope is further configured to provide insufflation media to the target site; and the processing circuitry is further configured to, in response to the determination that the pressure at the target site satisfies the specified condition, cause the endoscope to perform at least one of: provide an alert to a user to adjust a pressure of the insufflation media; or automatically adjust a pressure of the insufflation media.

In Example 4, the surgical system of any one of Examples 1-3 can optionally be configured such that: the processing circuitry is configured to analyze the pressure value by comparing the pressure value to a threshold pressure value; and the specified condition is that the pressure value exceeds the threshold pressure value.

In Example 5, the surgical system of any one of Examples 1-4 can optionally be configured such that: the alert data signal is configured to alert a user that the pressure at the target site may be too high.

In Example 6, the surgical system of any one of Examples 1-5 can optionally be configured such that: the endoscope is further configured to provide a flushing agent to the target site; and the processing circuitry is further configured to, in response to the determination that the pressure value exceeds the threshold pressure value, automatically reduce a flow rate of the flushing agent.

In Example 7, the surgical system of any one of Examples 1-6 can optionally be configured such that: the processing circuitry is configured to analyze the pressure value by comparing a rate of change of the pressure value to a threshold pressure slope value; the specified condition is that the rate of change of the pressure value exceeds the threshold pressure slope value; and the alert data signal is configured to alert a user that the pressure at the target site may be changing too quickly.

In Example 8, the surgical system of any one of Examples 1-7 can optionally be configured such that: the parameter is a pressure; the fiber Bragg grating is a first fiber Bragg grating; the sensing optical fiber includes a second fiber Bragg grating located proximal to the first fiber Bragg grating; the sensing controller is further configured to determine, from the optical frequency domain reflectometry, a first pressure value at the first fiber Bragg grating and a second pressure value at the second fiber Bragg grating; the processing circuitry is further configured to: analyze the first pressure value and the second pressure value by comparing a difference between the first pressure value and the second pressure value to a threshold pressure difference; and determine, from the analysis of the first pressure value and the second pressure value, that the pressure at the target site exceeds a threshold pressure difference; and the alert data signal is configured to alert a user that an insufflation media channel may be blocked.

In Example 9, the surgical system of any one of Examples 1-8 can optionally be configured such that: the parameter is a temperature; and the parameter value is a temperature value.

In Example 10, the surgical system of any one of Examples 1-9 can optionally be configured such that: the processing circuitry is configured to analyze the temperature value by comparing the temperature value to a threshold temperature value; and the specified condition is that the temperature value exceeds the threshold temperature value.

In Example 11, the surgical system of any one of Examples 1-10 can optionally be configured such that: the alert data signal is configured to alert a user that the temperature at the target site may be too high.

In Example 12, the surgical system of any one of Examples 1-11 can optionally be configured such that: the endoscope is further configured to provide therapeutic laser light to the target site; and the processing circuitry is further configured to, in response to the determination that the temperature value exceeds the threshold temperature value, automatically reduce a power of the therapeutic laser light.

In Example 13, the surgical system of any one of Examples 1-12 can optionally be configured such that: the endoscope is further configured to provide a flushing agent to the target site; and the processing circuitry is further configured to, in response to the determination that the temperature value exceeds the threshold temperature value, automatically increase a flow rate of the flushing agent.

In Example 14, the surgical system of any one of Examples 1-13 can optionally be configured such that: the endoscope includes a cooling element configured to controllably cool the target site; and the processing circuitry is further configured to, in response to the determination that the temperature value exceeds the threshold temperature value, automatically activate the cooling element.

In Example 15, the surgical system of any one of Examples 1-14 can optionally be configured such that: the processing circuitry is configured to analyze the temperature value by comparing the temperature value to a threshold temperature value; the specified condition is that the temperature value is below the threshold temperature value; the endoscope includes a heating element configured to controllably heat the target site; and the processing circuitry is further configured to, in response to the determination that the temperature value is below the threshold temperature value, automatically activate the heating element.

In Example 16, the surgical system of any one of Examples 1-15 can optionally be configured such that: the processing circuitry is configured to analyze the temperature value by comparing a rate of change of the temperature value to a threshold temperature slope value; the specified condition is that the rate of change of the temperature value exceeds the threshold temperature slope value; and the alert data signal is configured to alert a user that the temperature at the target site may be changing too quickly.

In Example 17, the surgical system of any one of Examples 1-16 can optionally be configured such that: the fiber Bragg grating is a first fiber Bragg grating; the sensing optical fiber includes a second fiber Bragg grating located proximal to the first fiber Bragg grating; the sensing controller is further configured to determine, from the optical frequency domain reflectometry, a first temperature value at the first fiber Bragg grating and a second temperature value at the second fiber Bragg grating; and the processing circuitry is further configured to: analyze the first temperature value and the second temperature value; and determine, from the analysis of the second temperature value, that a temperature at a component of the endoscope satisfies a specified condition.

In Example 18, a surgical system can comprise: an endoscope configured to extend distally toward a target site and provide insufflation media to the target site; a sensing optical fiber extending to a distal portion of the endoscope and including a fiber Bragg grating disposed at a distal portion of the sensing optical fiber; a sensing controller configured to: direct sensing light distally along the sensing optical fiber such that at least some of the sensing light reflects from the fiber Bragg grating as reflected light; perform optical frequency domain reflectometry on the reflected light; and determine, from the optical frequency domain reflectometry, a pressure value at the fiber Bragg grating; and processing circuitry configured to: compare the pressure value to a threshold pressure value; determine, from the comparison, that the pressure value exceeds a threshold pressure value; and in response to the determination that the that the pressure value exceeds the threshold pressure value, cause the endoscope to perform at least one of: provide an alert to a user to reduce a pressure of the insufflation media; or automatically reduce a pressure of the insufflation media.

In Example 19, the surgical system of Example 18 can optionally be configured such that: the endoscope is further configured to provide a flushing agent to the target site; and the processing circuitry is further configured to, in response to the determination that the pressure value exceeds the threshold pressure value, automatically reduce a flow rate of the flushing agent.

In Example 20, a surgical system can comprise: an endoscope configured to extend distally toward a target site, provide therapeutic laser light to the target site, and provide a flushing agent to the target site, the endoscope including a cooling element configured to controllably cool the target site; a sensing optical fiber extending to a distal portion of the endoscope and including a fiber Bragg grating disposed at a distal portion of the sensing optical fiber; a sensing controller configured to: direct sensing light distally along the sensing optical fiber such that at least some of the sensing light reflects from the fiber Bragg grating as reflected light; perform optical frequency domain reflectometry on the reflected light; and determine, from the optical frequency domain reflectometry, a temperature value at the fiber Bragg grating; and processing circuitry configured to: compare the temperature value to a threshold temperature value; determine, from the comparison, that the temperature value exceeds a threshold temperature value; and in response to the determination that the that the temperature value exceeds the threshold temperature value cause the endoscope to perform at least one of: provide an alert to a user that the temperature at the target site may be too high; automatically reduce a power of the therapeutic laser light; automatically increase a flow rate of the flushing agent; or automatically activate the cooling element.

1. A surgical system, comprising:
an endoscope configured to extend distally toward a target site;
a sensing optical fiber extending to a distal portion of the endoscope and including a first fiber Bragg grating disposed at a distal portion of the sensing optical fiber and a second fiber Bragg grating located proximal to the first fiber Bragg grating;
a sensing controller configured to:
direct sensing light distally along the sensing optical fiber such that at least some of the sensing light reflects from the first fiber Bragg grating and the second fiber Bragg grating as reflected light;
perform optical frequency domain reflectometry on the reflected light;
determine, from the optical frequency domain reflectometry, a first pressure value at the first fiber Bragg grating; and
determine, from the optical frequency domain reflectometry, a second pressure value at the second fiber Bragg grating; and
processing circuitry configured to:
analyze the first pressure value and the second pressure value by comparing a pressure difference between the first pressure value and the second pressure value to a threshold pressure difference;
determine, from the analysis of the first pressure value and the second pressure value, that the pressure difference exceeds the threshold pressure difference; and
generate an alert data signal in response to the determination that the pressure difference exceeds the threshold pressure difference, the alert data signal being configured to alert a user that an insufflation media channel may be blocked.

2. (canceled)

3. The surgical system of claim 1, wherein:
the endoscope is further configured to provide insufflation media to the target site; and
the processing circuitry is further configured to, in response to the determination that the pressure difference exceeds the threshold pressure difference, cause the endoscope to perform at least one of:
provide an alert to a user to adjust a pressure of the insufflation media; or automatically adjust a pressure of the insufflation media.

4-5. (canceled)

6. The surgical system of claim 1, wherein:
the endoscope is further configured to provide a flushing agent to the target site; and
the processing circuitry is further configured to, in response to the determination that the pressure difference exceeds the threshold pressure difference, automatically reduce a flow rate of the flushing agent.

7. A surgical system, comprising:
an endoscope configured to extend distally toward a target site;
a sensing optical fiber extending to a distal portion of the endoscope and including a fiber Bragg grating disposed at a distal portion of the sensing optical fiber;
a sensing controller configured to:
direct sensing light distally along the sensing optical fiber such that at least some of the sensing light reflects from the fiber Bragg grating as reflected light;
perform optical frequency domain reflectometry on the reflected light; and
determine, from the optical frequency domain reflectometry, a pressure value at the fiber Bragg grating; and
processing circuitry configured to:
analyze the pressure value;
determine, from the analysis of the pressure value, that a pressure at the target site satisfies a specified condition; and
generate an alert data signal in response to the determination that the pressure at the target site satisfies the specified condition,
wherein:
the processing circuitry is configured to analyze the pressure value by comparing a rate of change of the pressure value to a threshold pressure slope value;
the specified condition is that the rate of change of the pressure value exceeds the threshold pressure slope value; and
the alert data signal is configured to alert a user that the pressure at the target site may be changing too quickly.

8-17. (canceled)

18. A surgical system, comprising:
an endoscope configured to extend distally toward a target site and provide insufflation media to the target site;
a sensing optical fiber extending to a distal portion of the endoscope and including a first fiber Bragg grating disposed at a distal portion of the sensing optical fiber and a second fiber Bragg grating located proximal to the first fiber Bragg grating;
a sensing controller configured to:
direct sensing light distally along the sensing optical fiber such that at least some of the sensing light reflects from the first fiber Bragg grating and the second fiber Bragg grating as reflected light;
perform optical frequency domain reflectometry on the reflected light;
determine, from the optical frequency domain reflectometry, a first pressure value at the first fiber Bragg grating; and
determine, from the optical frequency domain reflectometry, a second pressure value at the second fiber Bragg grating; and
processing circuitry configured to:
compare a pressure difference between the first pressure value and the second pressure value to a threshold pressure difference;
determine, from the comparison, that the pressure difference exceeds the threshold pressure difference; and
in response to the determination that the pressure difference exceeds the threshold pressure difference, cause the endoscope to perform at least one of:
provide an alert to a user to reduce a pressure of the insufflation media; or
automatically reduce a pressure of the insufflation media.

19. The surgical system of claim 18, wherein:
the endoscope is further configured to provide a flushing agent to the target site; and
the processing circuitry is further configured to, in response to the determination that the pressure difference exceeds the threshold pressure difference, automatically reduce a flow rate of the flushing agent.

20. (canceled)
